(19) United States
(12) Patent Application Publication
(10) Pub. No.: US 2017/0224790 A1
(43) Pub. Date: Aug. 10, 2017

(54) FUNGAL GLUCOSYLCERAMIDE AS A VACCINE FOR FUNGAL INFECTIONS

(71) Applicants: KOHJIN LIFE SCIENCES CO., LTD., Tokyo (JP); THE RESEARCH FOUNDATION FOR THE STATE UNIVERSITY OF NEW YORK, Albany, NY (US)

(72) Inventors: Maurizio DEL POETA, Stony Brook, NY (US); Visesato MOR, Stony Brook, NY (US); Toshiya SATO, Chiba (JP); Tomohiro NAKAGAWA, Oita (JP)

(73) Assignees: KOHJIN LIFE SCIENCES CO., LTD., Tokyo (JP); THE RESEARCH FOUNDATION FOR THE STATE UNIVERSITY OF NEW YORK, Albany, NY (US)

(21) Appl. No.: 15/515,318

(22) PCT Filed: Sep. 30, 2015
(86) PCT No.: PCT/JP2015/004972

§ 371 Date: Mar. 29, 2017

Related U.S. Application Data
(60) Provisional application No. 62/058,622, filed on Oct. 1, 2014.

(51) Int. Cl. A61K 39/00

(52) U.S. Cl. CPC A61K 39/0002; A61K 2039/54; A61K 2039/545; A61K 2039/58

(57) ABSTRACT

The present invention features compositions that include a fungal glucosylceramide (GlcCer) purified from a non-pathogenic fungus (e.g., Candida utilis) and, optionally, an adjuvant. The invention also features methods of treating a patient who has a fungal disease and methods of preventing a fungal disease in a subject by administration of these compositions. Also within the scope of the invention are methods of formulating a fungal vaccine by: (a) providing a fungal glucosylceramide isolated from a non-pathogenic fungus; and (b) combining the fungal glucosylceramide with an adjuvant in a physiologically acceptable excipient.

### TECHNICAL FIELD

The present invention relates to an antigenic fungal glucosylceramide, compositions that include the glucosylceramide (for example, vaccines that can be used to treat or prevent fungal disease), and methods of making and using such compositions.

### BACKGROUND ART

Fungal infections pose a significant threat to public health. Fungi are common in the environment, as they can thrive in soil, on plants and trees, on innate surfaces, and on animate objects, including human skin. Despite the availability of antifungal agents, morbidity and mortality from invasive fungal infections remain high, particularly in critically ill patients. For reviews, see Enoch et al. (J. Medicinal Microbiol. 55:809-818, 2006) and Spellberg (Medicine Reports 3:13, 2011). Successfully eliminating fungal pathogens following prophylactic or therapeutic immunization depends in large part on the ability of the host's immune system to become appropriately activated in response to the immunization and to mount an effective response that does not significantly damage healthy tissue. A need exists for the development of, and improvement of, fungal vaccines.

### SUMMARY

The present invention is based, in part, on our studies indicating that administration of glucosylceramide purified from a non-pathogenic fungus is protective against pathogenic fungi. More specifically, our studies have shown that intraperitoneal administration of glucosylceramide purified from the non-pathogenic fungus Candida utilis (Torula yeast) significantly reduces the dissemination of Cryptococcus neoformans from the lung to the brain in mice, thus preventing the development of life-threatening meningoencephalitis. The present compositions not only induce active immunity, but also do so by virtue of a lipid antigen. In contrast, most vaccines are comprised of proteins or peptides. Because the source of the antigen can be a non-pathogenic fungus, we expect the methods of treatment disclosed herein will be protective against many, and possibly all, pathogenic fungi. Accordingly, in a first aspect, the invention features compositions that include a fungal glucosylceramide (GlcCer) purified from a non-pathogenic fungus (e.g., Candida utilis) and an adjuvant (e.g., 2-hydroxypropyl-β-cyclodextrin (HP-β-CD), Freund's complete adjuvant or Freund's incomplete adjuvant).

In a second aspect, the present invention features methods of treating a patient who has a fungal disease by administering to the patient a composition that includes a fungal glucosylceramide (e.g., a fungal glucosylceramide isolated from a non-pathogenic fungus). We expect that a broad range of fungal diseases can be treated, including those caused by infection with a fungus of the genus Absidia, Alternaria, Aspergillus, Basidiobolus, Bipolaris, Candida, Cladosporium, Cryptococcus, Curvularia, Epidermophyton, Klebsiella, Microsporum, Penicillium, Pneumocystis, Rhodotorula Saccharomyces, Stachybotrys, Trichophyton, Trichosporon, or Wangiella. The non-pathogenic fungus from which the GlcCer can be purified can be, for example, Candida utilis. The composition can be administered in a therapeutically effective amount, which is an amount that alleviates a sign or symptom of the fungal disease to an extent that the patient experiences relief and, preferably, complete relief. The composition can be administered daily until the patient is successfully treated. The composition can be formulated for topical administration or administration by an injection (e.g., a subcutaneous or intramuscular injection).

In a third aspect, the present invention features methods of preventing a fungal disease in a subject by administering to the subject a composition that includes a fungal glucosylceramide (e.g., a fungal glucosylceramide isolated from a non-pathogenic fungus). As with the methods of treatment, prophylactic methods can be used to prevent a diseased caused by infection with a fungus of the genus Absidia, Alternaria, Aspergillus, Basidiobolus, Bipolaris, Candida, Cladosporium, Cryptococcus, Curvularia, Epidermophyton, Klebsiella, Microsporum, Penicillium, Pneumocystis, Rhodotorula , Saccharomyces, Stachybotrys, Trichophyton, Trichosporon, or Wangiella. The non-pathogenic fungus from which the GlcCer is purified can be Candida utilis. The composition, regardless of the precise GlcCer antigen can further comprise an adjuvant, as described herein and known in the art of vaccine therapy. The step of administering the composition can occur at least twice, on a first occasion as a primary vaccination and on a second occasion (and subsequent occasions) as a booster immunization. As with methods of treatment, compositions administered for prophylaxis can be formulated for topical administration or administration by an injection (e.g., a subcutaneous or intramuscular injection).

In a fourth aspect, the present invention features methods of formulating a fungal vaccine by: (a) providing a fungal glucosylceramide isolated from a non-pathogenic fungus; and (b) combining the fungal glucosylceramide with an adjuvant in a physiologically acceptable excipient.

By “about” we mean within 10%, plus or minus, of a referenced value. For example, about 10 mg means 9-11 mg.

By “antigen” or “immunogen” we mean a substance that, upon administration to a subject (e.g., a human being), elicits the production of antibodies.

By “non-pathogenic fungus” we mean a fungus that does not cause disease in a subject.

By “pathogenic fungus” we mean a fungus that causes disease in a subject (e.g., a human being), whether the disease is commonly referred to as a disease per se or referred to as a disorder, condition, or the like. The pathogenic fungus can be one that causes disease in healthy subjects or it can be an opportunistic pathogen that causes infection in a subject who is immunocompromised.

By “prevention” we mean a forestalling of a clinical sign or symptom indicative of a fungal infection (e.g., a disease in a subject caused by a pathogenic fungus). Such forestalling includes the maintenance of normal physiological indicators in a subject at risk of fungal infection (e.g., maintenance of normal body temperature, weight, and psychological state), as well as a forestalling of lesions or other pathological manifestations of a fungal infection.

By “purified,” and with respect to the glucosylceremide, we mean at least or about 50% pure (e.g., at least 80%, 85%, 90%, 95%, 96%, 97%, 98%, 99% or more than 99% (e.g., about 99.5%) of a given formulation is glucosylceremide).

By “subject” we mean an individual living being, including a mammal such as a domesticated animal or a human being. Unless the context indicates otherwise, we use the terms “subject,” “individual,” and “patient” interchangeably. We may tend to use the term “patient” to refer to an individual living being who has been diagnosed as having a fungal infection.

By “treatment” we mean amelioration of a clinical sign or symptom experienced by a patient who has been diagnosed with a fungal infection. Similarly, methods of “treating” such a patient are methods of ameliorating a clinical sign or symptom experienced by the patient. The treatment or method of treating is successful when it arrests the progression of one or more of the signs or symptoms of a fungal disease and/or a patient experiences a reduction in the severity of the disease, a chronic complication of the disease, or an opportunistic fungal infection. In some patients, a treatment according to the present methods can inhibit or prevent the dissemination of fungi within a tissue or organ or from one tissue or organ to another. For example, in some patients, a treatment according to the present methods can inhibit or prevent the dissemination of Cryptococcus neoformans from the lung to the brain, thus preventing the development of life-threatening meningoencephalitis. Any of the methods of treatment described herein can also be expressed in terms of “use.” For example, the invention features use of a composition described herein in the preparation of a medicament or in the preparation of a medicament for the prevention or treatment of a fungal disease.

By “vaccine” we mean a composition that is administered to a subject (e.g., a human being) for the purpose of eliciting or boosting an immune response that will provide immunity against one or several diseases caused by a pathogenic fungus. The term encompasses compositions in a form suitable for administration to the subject as well as compositions in other forms (e.g., concentrated stock solutions and powdered or lyophilized forms that require further manipulation prior to administration). The subject can be an individual living being who does not currently have a fungal infection but who is at risk for developing such an infection.

We are aware of only a few vaccines that are effective against pathogenic fungi. Strategies have been pursued to elicit passive immunity as well. For instance, administration of a monoclonal antibody raised against glucosylceramide produced limited protection in mice infected with Cryptococcus neoformans, a yeast-like pathogen that can elicit cryptococcosis, which affects the central nervous system and can be fatal, especially in immunocompromised patients (Rodrigues et al., Clin. Vaccine Immunol., 14(10):1372-1376, 2007). While protection was observed in this mouse model, passive immunity is only partially protective against the fungus.

In the studies described below, we observed partial protection against the pathogenic fungus Cryptococcus neoformans following administration of GlcCer purified from the non-pathogenic fungus Candida utilis. The protection seemed to be mediated by antibodies against fungal GlcCer, and we therefore believe that fungal GlcCer can act as an antigen. Administering GlcCer with Freund's adjuvant improved the protection and seemed especially useful in decreasing the dissemination of fungal cells from the lung to the brain. With regard to mechanism, protection does not appear to be mediated by an opsonic effect of the anti-IgM antibodies.

### BRIEF DESCRIPTION OF DRAWINGS

FIGS. 1 and 2 illustrate the structures of purified GlcCer extracted from Candida utilis.

FIG. 1 illustrates GlcGer with 4,8-sphingadienine (d18:2) sphingoid base,

FIG. 2 illustrates GlcCer with 9-methyl-4,9-sphingadienine (d19:2) sphingoid base. R=C9-C27.

FIG. 3 illustrates a schedule for daily glucosylceramide administration to mice as described in Example 3.

FIG. 4 is a graph charting the survival of the mice treated as shown in FIG. 3 and further described in Example 3.

FIGS. 5-7 are bar graphs charting the fungal tissue burden in Cryptococcus-infected mice untreated or treated with GlcCer as shown in FIG. 3 and described in Example 3.

FIG. 5 charts the fungal tissue burden in infected mice vaccinated with vehicle (Group 5; “Solvent”). Log 10 CFUs/organ (from left to right, brain, lung, liver, spleen, and kidney) is plotted in mice that survived for 22, 24, 27, and 29 days (mouse #1 (“Cn1”), mouse #3 (“Cn3”), mouse #4 (“Cn4”), and mouse #6 (“Cn6”), respectively). Cryptococcal cells were present in all organs tested.

FIG. 6 charts the fungal tissue burden in four infected mice (designated #7, #8, #9, and #10) that were vaccinated with GlcCer (Group 3) and survived for 90 days. Fungal cells were found in lung tissue harvested from all four mice, in liver tissue harvested from two mice, and in kidney tissue harvested from two mice.

FIG. 7 charts the fungal tissue burden in four infected mice vaccinated with GlcCer+Freund's adjuvant (Group 4) that survived for 90 days. Fungal cells were found in only lung tissue, which was the primary site of the infection.

FIGS. 8 and 9 are panels of photomicrographs of brain (FIG. 8) and lung (FIG. 9) stained with hematoxylin and eosin (H&E; left-hand photographs) and mucicarmine (right-hand photographs). The treatment groups are as labeled.

FIG. 10 is a bar graph illustrating the presence of IgM anti-GlcCer antibodies in the sera of mice that were treated as indicated; “GlcCer” indicates glucosylceramide, “FA” indicates Freund's Adjuvant, “Cn” indicates Cryptococcus neoformans, and “Sol” indicates “solvent”.

FIG. 11 is a schematic of an experimental treatment regime in which glucosylceramide is administered weekly, as described further in Example 4.

FIG. 12 is a graph plotting the survival of mice that were not treated in any way (Group 3); mice that were treated with saline and challenged with Cryptococcus neoformans (Group 2); and mice that were treated with GlcCer and Freund's Adjuvant (Group 1) prior to challenge with Cryptococcus neoformans (as described further in Example 4).

FIG. 13 is a pair of bar graphs illustrating the antibody response (the amounts of IgM antibodies produced in the various treatment groups are shown in the left-hand graph and the amounts of IgG antibodies are shown in the right-hand graph). The mice in this study were treated as illustrated in FIG. 11 and further described in Example 4. The results illustrate a robust IgM response but no detectable IgG response during the period of observation. It is possible that an IgG response will develop at a later time point, but these data indicate that protection is not due to IgG (since we observed protection before an IgG response was stimulated).

FIG. 14 is a schematic illustrating a schedule for administering glucosylceramide in the presence or absence of adjuvant to different strains of mice using different routes of administration, as described further in Example 5.

FIG. 15 is a panel of four bar graphs showing the levels of anti-GlcCer antibodies in blood collected from the mice treated as shown in FIG. 14 and described further in Example 5.

FIG. 16 is a pair of bar graphs depicting the results of our study of phagocytosis of cryptococcus by macrophages in the presence of anti-GlcCer IgM or anti-GXM IgG antibodies.

FIG. 17 is a schematic illustrating the basic structure of glycosphingolipids. A long-chain sphingoid base backbone (distinguished from glycerolipids, which have a glycerol backbone) is linked to a fatty acid via an amide bond with the 2-amino group and to a polar head group at the C1 position via an ester bond, forming ceramide. The ceramide is linked to a sugar (glucose, galactose, or inositol) via a β-glycosidic bond between the hemiacetal group of the sugar and the C1 hydroxyl group of ceramide.

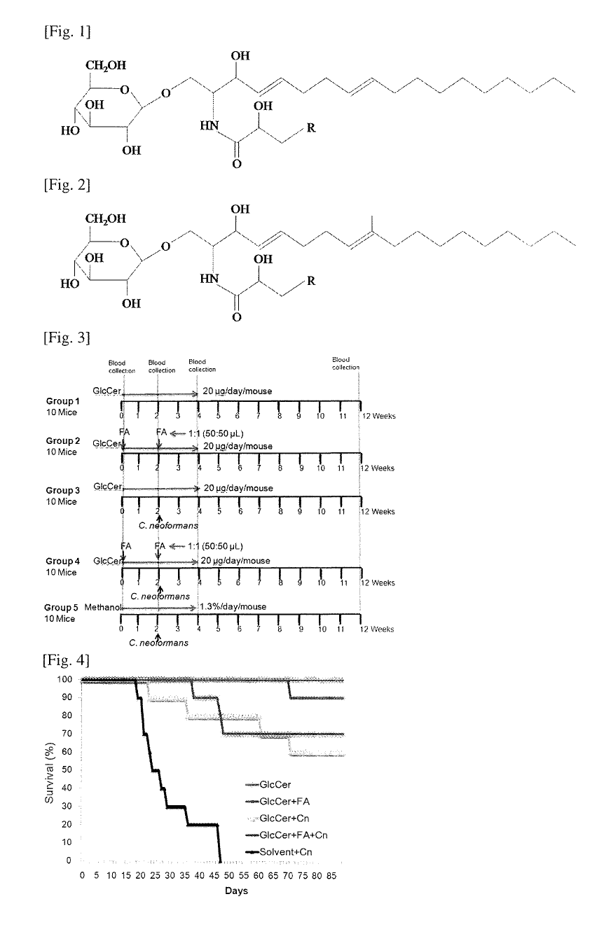

### DETAILED DESCRIPTION

Fungal glucosylceramide is an antigenic molecule that elicits the production of host antibodies in humans (Barr et al., Biochemistry 23:5589-5596, 1984; Barr and Lester, Biochemistry 23:5581-5588, 1984; Jimenez-Lucho et al., Infect. Immun. 58:2085-2090, 1990; Rodrigues, Infect. Immun. 68:7049-7060, 2000) or in mice (Toledo et al., Glycobiology 11:105-112, 2001, Rodrigues et al., Clin.Vaccine Immunol. 14:1372-1376, 2007). In these studies, antibody production was better when the anti-GlcCer antibodies were stimulated by GlcCer produced by the fungus during the infection (i.e., when an intact fungus was administered), than when antibody production was stimulated by GlcCer purified from plants and introduced exogenously. Antibody production was confirmed by adding patient serum to an ELISA plate onto which purified GlcCer had been absorbed. In addition, plant glucosylceramide (purified from soybean) elicits activation of local innate immunity in mice and protects them against the development of colon carcinoma (Symolon et al., J. Nutr. 134:1157-1161, 2004). Moreover, treatment of mice with plant glucosylceramide elicits protection against subsequent fungal infection (Umemura et al. Plant Cell Physiol. 43:778-784, 2002; Umemura, Plant Cell Physiol. 41:676-683, 2000; Koga, et al., J. Biol. Chem. 273:31985-31991, 1998). These observations suggest that glucosylceramide is a potent elicitor of both host innate and humoral immunity with consequent protection against fungal infections.

Glucosylceramide: Glucosylceramides (GlcCer; also called glucocerebrosides) include a ceramide, which is in turn composed of sphingosine (also referred to as a sphingoid base), a fatty acid, and a glucose residue. They are abundant in nature and found in plants, animals and fungi. The precise chemical structure of the GlcCer can vary depending on the type of fungi with which it is naturally associated. For example, the fatty acid attached to the sphingosine backbone can vary (the long-chain sphingoid base backbone is linked to the fatty acid via an amide bond with the 2-amino group). The fatty acid can include 4-28 carbon atoms, with short-chain fatty acids having less than six carbon atoms; medium-chain fatty acids having 6-12 carbon atoms; long-chain fatty acids having 13-21 carbon atoms; and very long-chain fatty acids having more than 22 carbon atoms. The carbon atoms in the sphingoid base or fatty acid can also be saturated or unsaturated and hydroxylated or non-hydroxylated. A glucosylceramide having any of these characteristics or any combination of these characteristics is within the scope of the present invention and can be included in the compositions and methods described herein. The invention further encompasses variants of naturally occurring GlcCer that have been methylated or acetylated, and these variants can be included in the present compositions and administered as described herein. Glucosylceramides play a role in fungal cell division, alkaline tolerance, hyphal formation, and spore germination. Thus, they are thought to be important in regulating fungal virulence. As GlcCer is a hydrophobic lipid, it is primarily localized in membranes.

The glucosylceramides in the present compositions (e.g., pharmaceutical compositions and vaccines) can be isolated from essentially any fungus, including many pathogenic fungi such as Candida utilis, Pichia pastoris, and others. The chemical structure of fungal GlcCer is different from the chemical structure of GlcCer expressed by various plants and mammals (Del Poeta, Plos Pathogen 2014), and GlcCer isolated or purified from various sources will naturally vary in, for example, the ways described above (the length of the carbon chain, the degree of saturation, and the degree of hydroxylation). Fungal GlcCer has a sphingosine backbone that is desaturated at position 8 by the delta-8-desaturase (S1d8) and methylated at position 9 by the delta-9-methyl transferase (Smt1). These two enzymes are only present in fungi; they are not known to be present in plant or in mammalian cells. Thus, the biochemical structure of fungal GlcCer is unique (Del Poeta, Plos Pathogens 2014), and we believe fungal GlcCer (e.g., purified from a non-pathogenic fungus) will stimulate the host immune response more strongly than GlcCer purified from plants. In fungal GlcCer, the ceramide backbone can be linked to 2-hydroxy-octadecanoic acid, occasionally with a trans bond in position 3. FIG. 17 illustrates the basic structure of glycosphingolipids.

The glucosylceramides in the present compositions (e.g., vaccines and pharmaceutical compositions useful for treatment) can be synthesized or purified from any non-pathogenic fungi. To purify useful glucosylceramides, one can, for example: (a) provide a yeast residue; (b) extract the residue with ethanol; (c) filter the extract; (d) saponify the filtered solution (e.g., by alkali hydrolysis of triglycerides and phospholipids); and (e) neutralize the saponified solution (e.g., with an acid). The salt formed under neutralization can be removed by filtration, and acetone precipitation can be used to further fractionate GlcCer. Silica gel chromatography can be conducted subsequently and repeatedly (e.g., twice) to purify GlcCer from the simple lipids, and preparative TLC can be used in the final step to prepare the purified GlcCer sample. The HPLC purity of the GlcCer sample can be above 98% compared to the GlcCer standard from soy. This method is described further in WO 2012/150683, as are analysis methods such as dry weight, TLC and HPLC. To purify GlcCer for formulation and use as described herein, one can consult the teaching of WO 2012/50683 and, also, the Examples below.

Subjects and patients amenable to the methods of preventing and treating fungal disease: Although all subjects are amenable to the methods of preventing fungal disease described herein, certain subjects are particularly amenable due to a predisposition to infection. Subjects who are particularly amenable include individuals who are immunocompromised due to, for example, infection with an immunodeficiency virus (e.g., HIV), immunosuppressive therapy, advanced age or premature birth. Other particularly amenable subjects include those undergoing an organ transplant (e.g., solid-organ transplantation), a blood transfusion, or bone marrow transplantation; subjects undergoing surgery, particularly a major surgery; subjects who have azotemia, diabetes mellitus, bronchiectasis, emphysema, tuberculosis, lymphoma, leukemia, or another type of cancer; subjects who have been burned or experienced another significant trauma; subjects with a history of susceptibility to a fungal infection; the very young (e.g., humans under about two years of age); the elderly (e.g., human over about 65 years of age); and subjects residing or working in an environment that is conducive to fungal infection.

The protection afforded by the prophylactic methods can stave off infection by one or more pathogenic fungi, and subjects amenable to the methods of treatment described herein can be treated for infection by one or more pathogenic fungi. The pathogenic fungi include species of Absidia, species of Alternaria, species of Aspergillus (e.g., A. flavatus, A. flavus, A. fumigatus, A. glaucus, A. nidulans, A. niger, A. sydowi, and A. terreus), species of Bipolaris, species of Candida (e.g., C. albicans, C. enolase, and C. glabrata, C. guilliermondi, C. krusei, C. kusei, C. lusitaniae, C. parakwsei, C. parapsilosis, C. pseudotropicalis, C. stellatoidea, and C. tropicalis), species of Cladosporium, species of Cryptococcus (e.g., C. albidus, C. gattii, C. laurentii, and C. neoformans), species of Histoplasma (e.g., H. capsulatum), species of Curvularia, species of Klebsiella (e.g., K. pneumoniae), species of Pneumocystis (e.g., P. carinii and P. jirovecii), species of Saccharomyces (e.g., S. boulardii, S. cerevisiae, and S. pombe), species of Trichosporon (e.g., T. beigelii), species of Rhodotorula, the Zygomycetes, hyaline moulds (e.g., Fusarium and Scedosporium species (e.g., S. apiosperum and S. prolificans)), species of Stachybotrys (e.g., S. chartarum), species of Penicillium (e.g., P. marnaeffei), and a wide variety of dematiaceous fungi. The fungal GlcCer included in the present compositions and formulations may also be obtained from a dermatophyte, including any species of the genera Microsporum, Epidermophyton, or Trichophyton (e.g., E. floccusum, M. audouini, M. canis, M. distortum, M. equinum, M. gypsum, M. nanum, T. concentricum, T. equinum, T. gallinae, T. gypseum, T. megnini, T. mentagrophytes, T. quinckeanum, T. rubrum, T. schoenleini, T. tonsurans, T. verrucosum, T. verrucosum var. album, var. discoides, var. ochraceum, T. violaceum, and/or T. faviforme. Others include species of Basidiobolus, Blastomyces dermatidis, Blastoschizomyces capitatus, species of Cladosporium (e.g., C. carrionii), Coccidioides immitis, species of Conidiobolus, species of Cunninghamella, species of Curvularia, species of Fonsecaea, Geotrichum clavatum, species of Helminthosporium, species of Malassezia, species of Monolinia, species of Mortierella, species of Mucor, species of Paecilomyces, Paracoccidioides brasiliensis, species of Pitliomyces, Pityrosporum ovale, Pythiumn insidiosum, species of Rhizoctonia, species of Rhizopus, species of Saksenaea, species of Sporothrix (e.g., S. schenckii), Toxoplasma gondii, and species of Wangiella.

Formulations and dosing: Fungal GlcCer can be formulated as a vaccine preparation intended for prophylaxis or as a therapeutic/medicament for the treatment of established fungal infections. The vaccine preparations may include an adjuvant, and the adjuvant can be 2-hydroxypropyl-β-cyclodextrin (HP-β-CD), Freund's complete adjuvant or Freund's incomplete adjuvant.

Various routes of administration and administration schedules can be employed. Our studies to date indicate that daily administration of GlcCer is slightly more efficacious than weekly administration. The compositions can be prepared for injection (e.g. as liquid solutions or suspensions). The invention also encompasses, however, solid forms that can be administered orally or dissolved or suspended in a liquid vehicle prior to injection. Administration will generally be parenteral (e.g., by injection, either subcutaneously, intraperitoneally, intravenously or intramuscularly). The compositions can also be administered into a lesion or absorbed through the skin or a mucous membrane. Thus, the invention features spray formulations, including formulations that can be administered by insufflation to the nasal passages, the lung, and tissues therebetween, suppositories, and transdermal or transcutaneous patches.

Preferably, the compositions of the invention are sterile, and they may include buffers to stabilize the pH generally around pH 7.0 (e.g., at a pH between about 6.0 and 8.0). Where the compositions include an aluminium hydroxide salt, the buffer is preferably a histidine buffer. The compositions may further include one or more of a detergent (e.g., a Tween, such as Tween 80) at low levels (e.g., <0.01%), a sugar alcohol (e.g., mannitol), and a preservative.

Optimum doses of individual antigens can be assessed empirically. The quantity to be administered, both according to the number of treatments and the amount of the antigen, can depend on the subject to be treated, the capacity of the subject's immune system to synthesize antibodies, and the degree of protection desired. In general, however, based on animal studies, we anticipate that 1-2 mg/kg/day should provide the desired protection.

The methods of treating or preventing fungal disease can be carried out according to a single dose schedule or a multiple dose schedule (e.g., with a primary dosage formulation being administered before one or more subsequent “boosters”). Where multiple doses are administered, the various doses may be given by the same or different routes of administration (e.g., an intravenous prime and a mucosal boost). We expect administration of more than one dose to be particularly useful in immunologically naive patients. Multiple doses can be administered at least or about 1 week apart (e.g., about 2, 3, 4, 6, 8, 10, 12, or 16 week apart). Annual boosters may be used for continued protection. In the case of chronic infection, administration should continue until at least clinical symptoms or laboratory tests indicate that the viral infection has been eliminated or substantially abated and for a period thereafter.

In some embodiments, the GlcCer antigen can be administered within a liposome according to methods for liposomal formulation that are well known in the art.

### EXAMPLES

### Example 1: Purification of Glucosylcerebrosides

Ethanol Extraction and Alkali Hydrolysis: 800 g of dried-yeast was extracted with 1.6 liters of 95% EtOH for 10 hrs at 60° C. with stirring. The extract was then separated from the yeast cells by paper filtration, and the resulting filtered solution was heated to 40° C. and 10 N KOH aq. was added to the final concentration of 0.4 N to start alkali hydrolysis. Saponification by alkali hydrolysis was carried out for 2 hrs at 40° C. with stirring. The extract was neutralized to pH 7 with 1 N HCl and KCl crystal formed under neutralization was removed by paper filter. The filtered solution was dried by a vacuum evaporator, and a part of the dried material was used to analyze dry weight and GlcCer content with HPLC.

Acetone Precipitation: The dry material prepared as just described was dissolved with 100 ml chloroform:methanol (2:1). Three (3) liters of acetone was added and mixed, and the resulting solution was left at −20° C. for 4 hrs before being centrifuged at 5,000 rpm for 10 minutes at −20° C. The supernatant was discarded, and the precipitate containing GlcCer was collected. A part of the precipitate was used to analyze dry weight and GlcCer content.

Silica Gel Chromatography: Silica gel (Iatrobeads) was purchased from Mitsubishi Chemical Medience and reconditioned at 120° C. for 2 hours prior to use. 60 g of reconditioned silica gel was suspended in chloroform then packed onto a column. The wet volume of the silica gel was measured to be 100 ml. The column was equilibrated with 200 ml of chloroform before loading the sample. An acetone precipitate containing GlcCer was dissolved with 5 ml chloroform and loaded onto the column, which was then washed with 300 ml chloroform (the elutant was discarded). The column was then washed with 400 ml chloroform:acetone (2:8; and the elutant was again discarded).

400 ml chloroform:acetone (1:9) was pumped into the column, and fractions were monitored by TLC. The fractions containing GlcCer were collected. 400 ml of acetone was then pumped into the column, and the fractions containing GlcCer were collected. GlcCer fractions from the above steps were mixed together and dried by a vacuum evaporator. A part of the dried material was used to analyze dry weight and GlcCer content. The above silica gel chromatography was repeated once more to further purify GlcCer.

Preparative TLC: Preparative TLC (silica gel 60, glass plate PLC, 20 cm×20 cm, 2 mm thickness) was purchased from Merck Millipore Japan. The dried material from the previous step was dissolved with 1 ml chloroform:methanol (7:3), and the sample was spotted on PLC and developed by chloroform:methanol:water (65:16:2). Silica gel containing GlcCer was collected and suspended in 50 ml chloroform:methanol (2:1) then filtered by cotton followed by a 0.5 μm PVDF filter. The filtered solution containing GlcCer was dried overnight with a vacuum evaporator. The total dry weight was measured and the purity of the GlcCer sample was analyzed by HPLC.

In Summary:

| Purification step | GlcCer | | Dry weight | | Purity | Yield |
|---|---|---|---|---|---|---|
| Yeast residue | 812 | mg | 800 | g | 0.10% | 100% |
| EtOH extraction and | 533 | mg | 35.5 | g | 1.50% | 66% |
| alkali hydrolysis | | | | | | |
| Acetone precipitation | 319 | mg | 2.2 | g | 15% | 39% |
| Silica gel | 192 | mg | 383 | mg | 50% | 24% |
| chromatography | | | | | | |
| (First time) | | | | | | |
| Silica gel | 125 | mg | 150 | mg | 83% | 15% |
| chromatography | | | | | | |
| (Second time) | | | | | | |
| Preparative TLC | 99.5 | mg | 100 | mg | 99.50% | 12% |

### Example 2: Detecting GlcCer Species

We analyzed GlcCer in the extracts obtained above by ESI-MS/MS (electrospray ionization mass spectrometry/mass spectrometry) using TSQ Quantum Ultra™ Triple Quadrupole Mass Spectrometer (Thermo Scientific, USA). Samples were suspended in a buffer containing 1 mM ammonium formate +0.2% formic acid in methanol. Samples were delivered to the MS by using direct syringe loop injection at the rate of 10 μl/min. Samples were analyzed as [M+H]⁺ in the positive ion mode. We used a source voltage of 4.5 kV and collision energies of 20V. All the GlcCer spectra (Table 1) were detected from m/z 200 to 1000. MS-MS profiles were generated using two different collision energies, 20 and 45V. We detected GlcCer species with 4,8-sphingadienine (d18:2) and 9-methyl-4,9-sphingadienine (d19:2) sphingoid base using parent ion scanning for the fragment of 262.2 and 276.2 respectively. These fragments result from the cleavage of amide linkage and subsequent dehydration.

TABLE 1

| | | | Ex- | | | | | | | |
|---|---|---|---|---|---|---|---|---|---|---|
| | | | actMo- | | | | | | | |
| S. | GlcCer | | lar | | Fatty | Batch | Batch | Batch | | |
| No. | species | Formula | mass | Sphingoid base | acid | 1 | 2 | 3 | Mean ± SEM | Remarks |
| 1 | d18:2/C12:0h | C36H67NO9 | 657.47977 | 4,8-Sphingadienine | C12:0h | 0.000 | 0.000 | 0.000 | 0.000 ± 0.000 | Below detection, |
| | | | | (d18:2) | | | | | | probably |
| | | | | | | | | | | false positive. |
| 2 | d18:2/C14:0h | C38H71NO9 | 685.51097 | 4,8-Sphingadienine | C14:0h | 0.000 | 0.000 | 0.000 | 0.000 ± 0.000 | Below detection, |
| | | | | (d18:2) | | | | | | probably |
| | | | | | | | | | | false positive. |
| 3 | d18:2/C16:0h* | C40H75NO9 | 713.54217 | 4,8-Sphingadienine | C16:0h | 0.505 | 1.915 | 0.000 | 0.807 ± 0.702 | Detected only in |
| | | | | (d18:2) | | | | | | Batch 1 and 2 |
| 4 | d18:2/C18:0h* | C42H79NO9 | 741.57337 | 4,8-Sphingadienine | C18:0h | 22.744 | 18.054 | 3.538 | 14.779 ± 7.081 | Delected in all |
| | | | | (d18:2) | | | | | | three batches |
| 5 | d18:2/C20:0h | C44H83NO9 | 769.60457 | 4,8-Sphingadienine | C20:0h | 0.000 | 0.000 | 1.440 | 0.480 ± 0.588 | Detected only |
| | | | | (d18:2) | | | | | | in Batch 3 |
| 6 | d18:2/C22:0h | C46H87NO9 | 797.63577 | 4,8-Sphingadienine | C22:0h | 0.000 | 0.000 | 0.000 | 0.000 ± 0.000 | Below detection, |
| | | | | (d18:2) | | | | | | probably |
| | | | | | | | | | | false positive. |
| 7 | d18:2/C24:0h | C48H91NO9 | 825.66697 | 4,8-Sphingadienine | C24:0h | 0.000 | 0.000 | 0.000 | 0.000 ± 0.000 | Below detection, |
| | | | | (d18:2) | | | | | | probably |
| | | | | | | | | | | false positive. |
| 8 | d18:2/C26:0h* | C50H95NO9 | 853.69817 | 4,8-Sphingadienine | C26:0h | 0.249 | 0.722 | 0.000 | 0.324 ± 0.259 | Dectected only in |
| | | | | (d18:2) | | | | | | Batch 1 and 2 |
| 9 | d18:2/C28:0h | C52H99NO9 | 881.72937 | 4,8-Sphingadienine | C28:0h | 0.000 | 0.000 | 0.000 | 0.000 ± 0.000 | Below detection, |
| | | | | (d18:2) | | | | | | probably |
| | | | | | | | | | | false positive. |
| 10 | d18:2/C30:0h | C54H103NO9 | 909.76057 | 4,8-Sphingadienine | C30:0h | 0.746 | 0.000 | 0.000 | 0.249 ± 0.304 | Delected only |
| | | | | (d18:2) | | | | | | in Batch 1 |
| 11 | d19:2/C12:0h | C37H69NO9 | 671.49537 | 9-Methyl-4,9- | C12:0h | 0.000 | 0.000 | 0.000 | 0.000 ± 0.000 | Below detection, |
| | | | | Sphingadienine | | | | | | probably |
| | | | | (d19:2) | | | | | | false positive. |
| 12 | d19:2/C14:0h | C39H73NO9 | 699.52657 | 9-Methyl-4,9- | C14:0h | 0.000 | 0.000 | 0.000 | 0.000 ± 0.000 | Below detection, |
| | | | | Sphingadienine | | | | | | probably |
| | | | | (d19:2) | | | | | | false postive. |
| 13 | d19:2/C16:0h* | C41H77NO9 | 727.55777 | 9-Methyl-4,9- | C16:0h | 5.705 | 2.810 | 1.742 | 3.419 ± 1.450 | Detected in all |
| | | | | Sphingadienine | | | | | | three batches |
| | | | | (d19:2) | | | | | | |
| 14 | d19:2/C18:0h* | C43H81NO9 | 755.58897 | 9-Methyl-4,9- | C18:0h | 70.049 | 76.497 | 93.094 | 79.880 ± 8.407 | Detected in all |
| | | | | Sphingadienine | | | | | | three batches |
| | | | | (d19:2) | | | | | | |
| 15 | d19:2/C20:0h | C45H85NO9 | 783.62017 | 9-Methyl-4,9- | C20:0h | 0.000 | 0.000 | 0.184 | 0.061 ± 0.075 | Below detection, |
| | | | | Sphingadienine | | | | | | probably |
| | | | | (d19:2) | | | | | | false positive. |
| 16 | d19:2/C22:0h | C47H89NO9 | 811.65137 | 9-Methyl-4,9- | C22:0h | 0.000 | 0.000 | 0.000 | 0.000 ± 0.000 | Below detection, |
| | | | | Sphingadienine | | | | | | probably |
| | | | | (d19:2) | | | | | | false positive. |
| 17 | d19:2/C24:0h | C49H93NO9 | 839.68257 | 9-Methyl-4,9- | C24:0h | 0.000 | 0.000 | 0.000 | 0.000 ± 0.000 | Below detection, |
| | | | | Sphingadienine | | | | | | probably |
| | | | | (d19:2) | | | | | | false postive. |
| 18 | d19:2/C26:0h | C51H97NO9 | 867.71377 | 9-Methyl-4,9- | C26:0h | 0.000 | 0.000 | 0.000 | 0.000 ± 0.000 | Below detection, |
| | | | | Sphingadienine | | | | | | probably |
| | | | | (d19:2) | | | | | | false positive. |
| 19 | d19:2/C28:0h | C53H101NO9 | 895.74497 | 9-Methyl-4,9- | C28:0h | 0.000 | 0.000 | 0.000 | 0.000 ± 0.000 | Below detection, |
| | | | | Sphingadienine | | | | | | probably |
| | | | | (d19:2) | | | | | | false positive. |
| 20 | d19:2/C30:0h | C55H105NO9 | 923.77617 | 9-Methyl-4,9- | C30:0h | 0.000 | 0.000 | 0.000 | 0.000 ± 0.000 | Below detection, |
| | | | | Sphingadienine | | | | | | probably |
| | | | | (d19:2) | | | | | | false postive. |

### Example 3: Administration of GlcCer and Subsequent Challenge with Cryptococci

We purchased four-week old female CBA/J mice from Jackson Laboratory and divided them into five groups, with 10 mice in each group (n=10). The mice in Groups 1 and 3 received an intraperitoneal (ip) injection of purified glucosylceramide (GlcCer) using the method illustrated above at 20 μg/mouse/day in a 100 μl final volume. Since the mice weighed approximately 25 g, the injection dose was 1.6 mg/kg/day. The lipid (GlcCer) was suspended in a solution made of 1.3% methanol in phosphate buffered saline (PBS). Thus, 100 μl of a solution of 1.3% methanol in PBS containing 20 μg of GlcCer was injected intraperitoneally in each mouse every day. The solution was stored at −20 ° C. between the injections. The mice in Groups 2 and 4 received 20 μg/mice of GlcCer+incomplete Freund's adjuvant (FA), and the mice in Group 5 received a vehicle-only control (1.3% methanol in PBS). After 2 weeks, the animals in Groups 3, 4 and 5 were challenged intranasally with 5×10⁵ cryptococcal cells (Cryptococcus neoformans) whereas the animals in Groups 1 and 2 received PBS. The mice were fed ad libitum and monitored for signs of discomfort or sickness, and their survival was recorded. At 0, 2, 4, and 12 weeks, blood was drawn from mice to check the presence of GlcCer antibody by ELISA.

As shown in FIG. 4, uninfected mice injected with GlcCer (Group 1) or GlcCer+FA (Group 2) exhibited survival rates of 100% and 90%, respectively. Amongst the infected groups, administration of GlcCer provided 60% protection (Group 3), and GlcCer+FA (Group 4) provided 70% protection. In contrast, the infected group receiving only the delivery vehicle (Group 5) experienced 100% mortality with an average survival rate of 25 days.

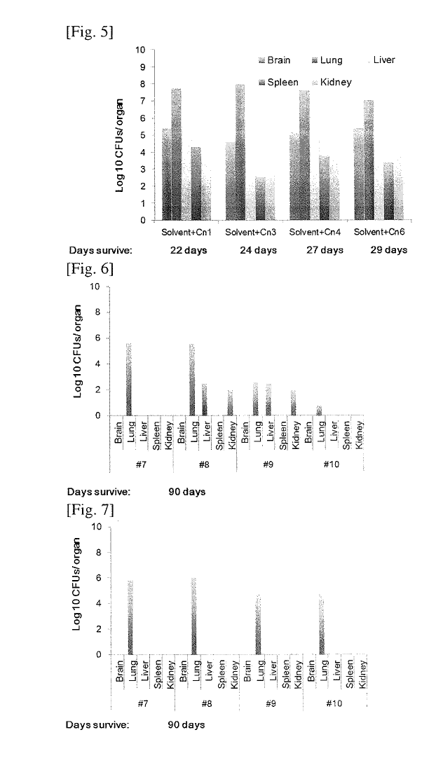

We assessed fungal tissue burden in the Cryptococcus-infected mice, and the results are illustrated in FIGS. 5-7. In infected mice vaccinated with vehicle only (Group 5), we found cryptococcal cells in all organs tested (brain, lung, liver, spleen and kidney; see FIG. 5). We also tested four infected mice vaccinated with GlcCer (Group 3) that survived for 90 days. We found fungal cells in the lung tissue harvested from each of the four mice, in the liver tissue of two mice, and in the kidney tissue of two mice. No cryptococcal cells were found in the brains or spleens of these animals (see FIG. 6). We also tested four infected mice vaccinated with GlcCer+Freund's adjuvant (Group 4) that survived for 90 days. Fungal cells were found in the lung only (the primary site of the infection, as fungal cells were injected intranasally). No cryptococcal cells were found in the brain, liver, kidney or spleen. These results suggest that the GlcCer+Freund's adjuvant formulation is the most effective in preventing fungal cells from disseminating from the lung to other tissues or organs. We also noted that the number of cryptococcal cells recovered from the lung after 90 days of infection in mice in either group 3 or 4 was approximately ˜5×10⁵ cells, which is similar to the number of cells that were injected at day 0. This indicates that vaccination with GlcCer or GlcCer+Freund's adjuvant arrests fungal cell proliferation in the lung.

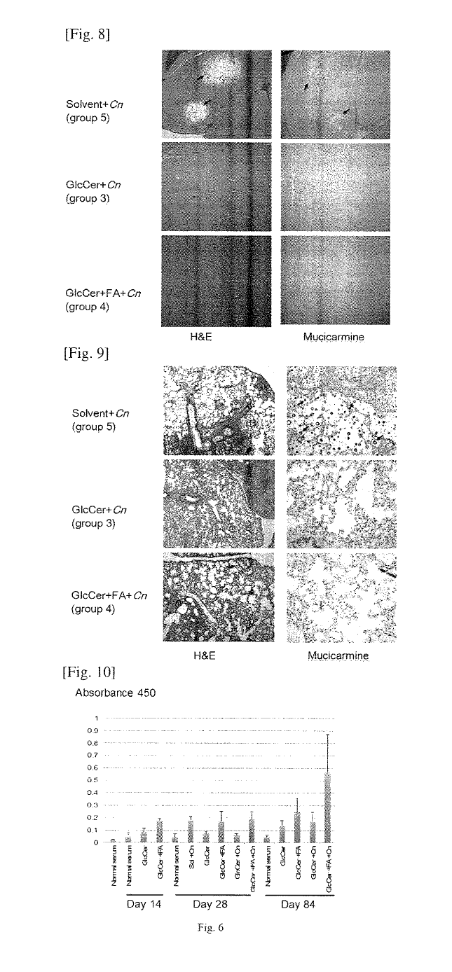

We examined brain and lung tissue harvested from mice in Groups 3, 4, and 5, and data from that study are shown in FIGS. 8 and 9. Brain tissue from mice infected with Cryptococcus neoformans but not treated (Group 5) showed extensive cryptococcal infiltration in the brain (see the arrows in FIG. 8) on day 25 of the infection. In contrast, brain tissue from mice vaccinated with either GlcCer alone (Group 3) or GlcCer+FA (Group 4) showed no cryptococcal cells after 90 days of infection. In lung tissue, we observed massive dispersion of cryptococcal cells in mice infected but not treated (arrows in FIG. 9; 25 days post-infection). In contrast, lungs from mice that were infected and treated with GlcCer (Group 3) or with GlcCer+FA (Group 4), showed no cryptococcal cells in the sections analyzed. The tissues were fixed in 10% formalin and paraffin embedded for microtome sectioning and staining. The photographs were taken through a ZEISS microscope.

We performed additional studies to detect anti-glucosylceramide antibody in mouse serum. We collected blood from the mice treated as illustrated in FIG. 3 on day 0, 14, 28 and 84 of the treatment regime. The blood was allowed to clot, and the serum was collected by centrifugation. Glucosylceramide antibody (IgM) in the mice serum was detected by a sandwich enzyme-linked immunosorbent assay (ELISA) using purified GlcCer as the antigen source. The reaction was stopped and absorbance was recorded at 450 nm using a FliterMax multiplate reader. We detected IgM anti-GlcCer antibodies in sera from the mice receiving GlcCer, and the highest levels were observed in mice immunized with GlcCer and FA at 84 days post-infection with Cryptococcus neoformans (FIG. 10).

At that same time point (84 days post-infection), we analyzed blood cell counts and performed tests for liver and kidney function. As shown in Table 2, there were no major changes in total leukocyte count, although infected mice showed higher white blood cell counts (particularly of neutrophils).

TABLE 2

| Leukocytes | Normal | Control | GlcCer | GlcCer + FA | GlcCer + Cn | GlcCer + FA + Cn |
|---|---|---|---|---|---|---|
| (Units) | range | (n = 3) | (n = 3) | (n = 3) | (n = 3) | (n = 3) |
| WBC (10\hat{ }3/μL) | 1.80-10.70 | 4.11 ± 0.56 | 3.40 ± 0.96 | 5.60 ± 10.00 | 5.10 ± 0.50 | 5.26 ± 0.64 |
| NE (10\hat{ }3/μL) | 0.10-2.40 | 0.40 ± 0.03 | 0.80 ± 10.00 | 0.59 ± 0.90 | 0.90 ± 0.06 | 1.44 ± 0.07 |
| LY (10\hat{ }3/μL) | 0.90-9.30 | 3.34 ± 0.52 | 3.42 ± 0.67 | 3.69 ± 0.45 | 3.38 ± 0.66 | 2.81 ± 0.50 |
| MO (10\hat{ }3/μL) | 0.00-0.40 | 0.32 ± 0.05 | 0.13 ± 0.03 | 0.11 ± 0.02 | 0.09 ± 0.02 | 0.07 ± 0.01 |
| EO (10\hat{ }3/μL) | 0.00-0.20 | 0.02 ± 0.03 | 0.10 ± 0.02 | 0.06 ± 0.00 | 0.09 ± 0.00 | 0.12 ± 0.03 |
| BA (10\hat{ }3/μL) | 0.00-0.20 | 0.01 ± 0.01 | 0 | 0.01 ± 0.00 | 0 | 0 |

WBC, White blood cell; NE, Neutrophiles; LY, lymphocytes; MO, Monocytes; EO, Eosinophiles; BA, Basophiles.

As shown in Table 3, there were no major changes in total erythrocyte or thrombocyte counts.

TABLE 3

| | Normal | Control | GlcCer | GlcCer + FA | GlcCer + Cn | GlcCer + FA + Cn |
|---|---|---|---|---|---|---|
| | range | (n = 3) | (n = 3) | (n = 3) | (n = 3) | (n = 3) |
| Erythrocytes | | | | | | |
| (Units) | | | | | | |
| RBC (10\hat{ }6/μL) | 6.36-9.42 | 7.33 ± 0.24 | 6.93 ± 0.97 | 7.98 ± 0.51 | 8.50 ± 0.56 | 8.10 ± 0.84 |
| Hb (g/dL) | 11.00-15.10 | 11.15 ± 0.07 | 11.80 ± 1.47 | 13.10 ± 1.10 | 13.70 ± 0.56 | 12.26 ± 1.35 |
| MCV (fL) | 45.40-60.30 | 53.67 ± 0.25 | 50.66 ± 6.43 | 47.00 ± 3.46 | 34.66 ± 23.28 | 45.33 ± 1.50 |
| MCH (pg) | 14.10-19.30 | 15.00 ± 0.30 | 17.06 ± 0.80 | 16.43 ± 1.30 | 26.10 ± 16.41 | 15.13 ± 0.23 |
| MCHC (g/dL) | 30.20-34.20 | 28.05 ± 0.45 | 34.06 ± 2.70 | 35.10 ± 0.70 | 28.33 ± 11.30 | 33.53 ± 1.15 |
| Thrombocytes | | | | | | |
| (Units) | | | | | | |
| PLT (10\hat{ }3/μL) | 592.00-2972.00 | 372.33 ± 221.24 | 624.33 ± 245.00 | 801.60 ± 185.20 | 493.00 ± 416.00 | 620.00 ± 107.00 |

RBC, Red blood cell; Hb, Haemoglobin; MCV, Mean corpuscularvol.; MCH, Mean corpuscular haemoglobin; MCHC, Mean corpuscular haemoglobin concentration; PLT, Platelets.

As shown in Table 4, there were no major changes in liver and kidney function:

TABLE 4

| Test | Normal | Control | GlcCer + Cn | GlcCer + FA + Cn | GlcCer + Cn | GlcCer + FA + Cn |
|---|---|---|---|---|---|---|
| (Units) | range | (n = 3) | (n = 3) | (n = 3) | (n = 3) | (n = 3) |
| ALP (U/L) | 35.00-101.00 | 114.33 ± 13.27 | 92.30 ± 12.20 | 77.00 ± 8.80 | 71.00 ± 12.71 | 68.66 ± 8 62 |
| ALT (U/L) | 17.00-32.00 | 28.00 ± 1.73 | 27.30 ± 8.00 | 23.66 ± 2.10 | 37.00 ± 15.71 | 27.33 ± 6 65 |
| AST (U/L) | 54.00-120.00 | 48.00 ± 5.56 | 116.60 ± 61.00 | 78.00 ± 14.90 | 108.66 ± 17.00 | 101.66 ± 17.92 |
| TBILI | 2.00-2.40 | 0.23 ± 0.05 | 0 13 ± 0.05 | 0.13 ± 0.05 | 0.16 ± 0.11 | 0.17 ± 0.12 |
| (mg/dL) | | | | | | |

ALP, Alkaline phosphatase; ALT, Alanine aminotransferase, AST, Aspartate aminotransferase; TBILI, Total bilirubin.

### Example 4: Weekly Administration of GlcCer

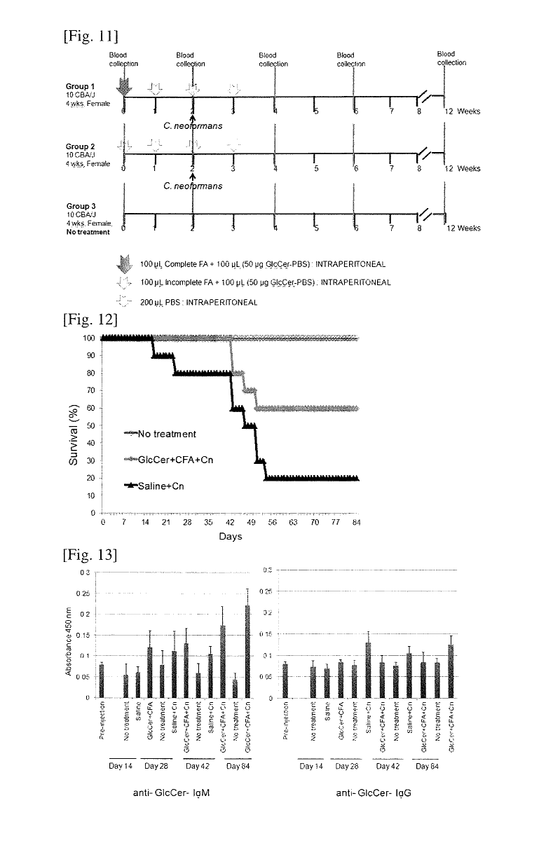

We purchased four-week old female CBA/J mice from Jackson Laboratory. On day 1, we injected the animals in Group 1 (n=10) intraperitoneally with 50 μg of GlcCer in the presence of complete Freund's adjuvant. We followed this with injections of 50 μg of GlcCer in the presence of incomplete Freund's adjuvant every week for 3 weeks. In this experiment, the mice of Group 2 (n=10) received only PBS, and the mice of Group 3 (n=10) were not treated in any way. Two weeks after the initial injection of GlcCer, the mice in Groups 1 and 2 were challenged with 5×10⁵ cryptococcal cells. The mice were fed ad libitum and monitored for survival. At 0, 2, 4, 6 and 12 weeks, we drew blood from the mice to check for the presence of anti-GlcCer antibodies. See FIG. 11. With regard to survival, the untreated mice (Group 3) survived as expected. The mice that were injected with GlcCer and Freund's adjuvant, as shown in FIG. 11 (Group 1) exhibited 60% protection when challenged with Cryptococcus neoformans whereas the mice that were treated with only PBS had a mortality rate of 80% upon challenge. See FIG. 12.

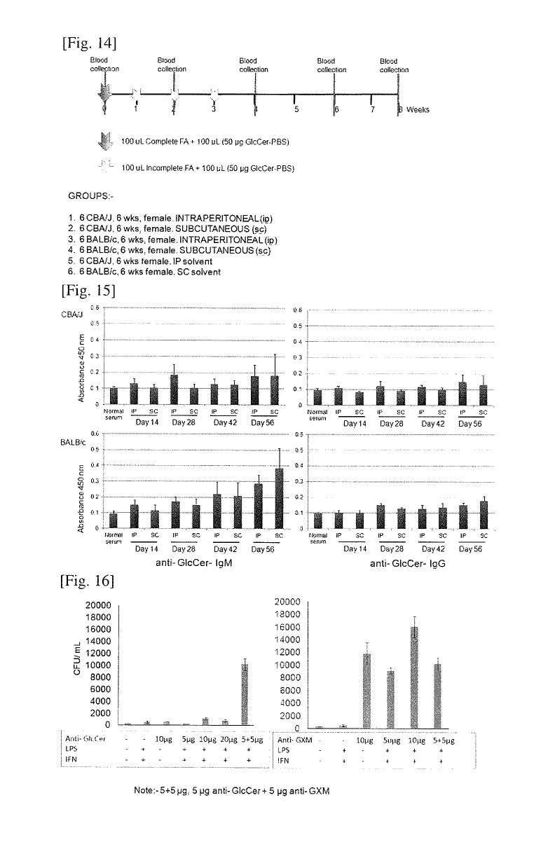

We performed studies to detect anti-GlcCer antibodies in serum from mice injected weekly with GlcCer (as described here and illustrated in FIG. 11). We collected blood on day 0 (pre-injection), and 14, 28, 42 and 84 days after initiation of the treatment program. Blood was allowed to clot and serum was collected by centrifugation. Glucosylceramide antibodies in the mouse serum were detected using a sandwich ELISA with purified GlcCer as the antigen source. IgM and IgG isotypes were determined by using IgM- or IgG-specific secondary antibodies. The reaction was stopped and absorbance recorded at 450 nm using a FliterMax multiplate reader. The results show that the administration of GlcCer stimulates an antibody response (IgM), particularly at day 84. See FIG. 13.

### Example 5: Administration of Glccer to Different Strains of Mice Using Different Routes of Administration

We purchased six-week old female CBA/J mice and six-week old female BALB/c mice from Jackson Laboratory. We divided the mice into six groups (n=6), with the CBA/J mice constituting Groups 1, 2, and 5, and the BALB/c mice constituting Groups 3, 4, and 6. On day 0, we injected the mice of Groups 1-4 with 50 μg of GlcCer in the presence of complete Freund's adjuvant; those in Groups 1 and 3 were injected intraperitoneally and those in groups 2 and 4 were injected subcutaneously. We injected the mice of Groups 5 and 6 with only the vehicles used for the treatment groups. One week after the initial treatment, we injected the mice in Groups 1-4 with 50 μg of GlcCer in the presence of incomplete Freund's adjuvant, and we repeated that administration weekly for a total of three weeks following the initial treatment. The Groups are summarized in Table 5:

| Group | Strain | Initial Treatment | Subsequent Treatment | Route |
|---|---|---|---|---|
| 1 | CBA/J | 50 μg GlcCer + | 50 μg GlcCer + | IP |
| | | complete FA | incomplete FA, | |
| | | | weekly for three weeks | |
| 2 | CBA/J | 50 μg GlcCer + | 50 μg GlcCer + | SC |
| | | complete FA | incomplete FA, | |
| | | | weekly for three weeks | |
| 3 | BALB/c | 50 μg GlcCer + | 50 μg GlcCer + | IP |
| | | complete FA | incomplete FA, | |
| | | | weekly for three weeks | |
| 4 | BALB/c | 50 μg GlcCer + | 50 μg GlcCer + | SC |
| | | complete FA | incomplete FA, | |
| | | | weekly for three weeks | |
| 5 | CBA/J | Intraperitoneal (IP) | IP vehicle | IP |
| | | vehicle | | |
| 6 | BALB/c | Subcutaneous (SC) | SC vehicle | SC |
| | | vehicle | | |

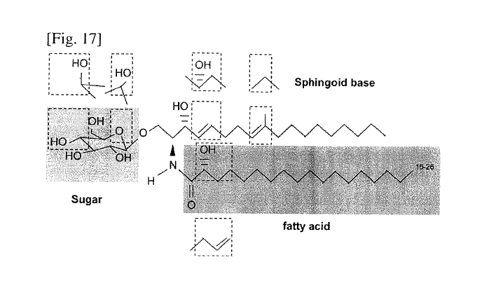

We collected blood on day 0 and 2, 4, 6, and 8 weeks after day 0 to check for the presence of antibodies that specifically bind GlcCer. None of the mice in this study were infected with Cryptococcus. The collected blood was allowed to clot and serum was collected by centrifugation. Anti-glucosylceramide antibodies were detected in the serum by a sandwich ELISA using purified GlcCer as the antigen source. IgM and IgG isotypes were detected using IgM- and IgG-specific secondary antibodies. The reaction was stopped and absorbance was recorded at 450 nm using a FliterMax multiplate reader. The results show the production of anti-GlcCer IgM antibody either when GlcCer was administered intraperitoneally or subcutaneously, particularly at 56 days after the first dose. There was no significant difference in antibody production between the two routes of administration. See FIG. 15.

To study the mechanism by which the anti-GlcCer antibodies affected cryptococcal cells, we co-cultured cryptococcal cells with the murine macrophage cell line J774. We grew the macrophages in a 96 well cell culture plate in DMEM containing 10% FCS at 37° C. in the presence of 5% CO₂ for 14 hours. We washed off the non-adhered cells with warm DMEM. Cryptococcal cells (10⁵ cells/200 μL) with a target cell ratio of 1:1 with J774 cells were opsonized with different concentrations of GlcCer antibodies (as shown in FIG. 16) and added to the J774 cells in the presence of lipopolysaccharide (LPS) and interferon (IFNγ). We performed a similar experiment with anti-GXM IgG antibody as a positive control. The cells were incubated at 37° C. in DMEM containing 10% FCS in the presence of 5% C0₂, for 2 hours to allow phagocytosis. After 2 hours, the non-phagocytosed Cn cells were washed off with warm DMEM. Macrophage cells were ruptured by the addition of sterile water and an aliquot was spread onto YPD-agar plate to determine the number of colony-forming units. We observed a slight increase in phagocytosis when Cn cells were opsonized with 10 μg or 20 μg of anti-GlcCer IgM antibody, but this increase was not significant. These results suggest that anti-GlcCer antibodies are providing protection against cryptococci through a mechanism that does not rely on stimulating phagocytosis and intracellular killing.

1. A composition comprising a fungal glucosylceramide (GlcCer) purified from a non-pathogenic fungus and an adjuvant.

2. The composition of claim 1, wherein the non-pathogenic fungus is Candida utilis.

3. A method of treating a patient who has a fungal disease, the method comprising administering to the patient a composition comprising a fungal glucosylceramide isolated from a non-pathogenic fungus.

4. The method of claim 3, wherein the fungal disease is caused by infection with a fungus of the genus Absidia, Alternaria, Aspergillus, Basidiobolus, Bipolaris, Candida, Cladosporium, Cryptococcus, Curvularia, Epidermophyton, Klebsiella, Microsporum, Penicillium, Pneumocystis, Rhodotorula , Saccharomyces, Stachybotrys, Trichophyton, Trichosporon, or Wangiella.

5. The method of claim 3, wherein the non-pathogenic fungus is Candida utilis.

6. The method of claim 3, wherein the method comprises administering the composition daily until the patient is successfully treated.

7. The method of claim 3, wherein the composition is formulated for topical administration or administration by an injection.

8. A method of preventing a fungal disease in a subject, the method comprising administering to the subject a composition comprising a fungal glucosylceramide isolated from non-pathogenic fungus.

9. The method of claim 8, wherein the fungal disease is caused by infection with a fungus of the genus Absidia, Alternaria, Aspergillus, Basidiobolus, Bipolaris, Candida, Cladosporium, Cryptococcus, Curvularia, Epidermophyton, Klebsiella, Microsporum, Penicillium, Pneumocystis, Rhodotorula , Saccharomyces, Stachybotrys, Trichophyton, Trichosporon, or Wangiella.

10. The method of claim 8, wherein the non-pathogenic fungus is Candida utilis.

11. The method of claim 8, wherein the composition further comprises an adjuvant.

12. The method of claim 8, wherein administering the composition occurs at least twice, on a first occasion as a primary vaccination and on a second occasion as a booster immunization.

13. The method of claim 3, wherein the composition is formulated for topical administration or administration by an injection.

14. A method of formulating a fungal vaccine, the method comprising
(a) providing a fungal glucosylceramide isolated from a non-pathogenic fungus; and
(b) combining the fungal glucosylceramide with an adjuvant in a physiologically acceptable excipient.
